(19) United States
(12) Patent Application Publication
(10) Pub. No.: US 2011/0245671 A1
(43) Pub. Date: Oct. 6, 2011

(54) ULTRASOUND IMAGING METHOD AND APPARATUS

(75) Inventor: Tomoo SATO, Ashigara-kami-gun (JP)

(73) Assignee: FUJIFILM Corporation, Tokyo (JP)

(21) Appl. No.: 13/023,177

(22) Filed: Feb. 8, 2011

(30) Foreign Application Priority Data
Mar. 31, 2010 (JP) 2010-083714

(51) Int. Cl. A61B 8/14

(52) U.S. Cl. 600/443

(57) ABSTRACT

An ultrasound imaging method comprises the steps of: transmitting respective ultrasonic waves from a plurality of ultrasound transducers, arranged in an array, of an ultrasound probe; determining respective propagation times of the ultrasonic waves inside a cranium corresponding to each ultrasound transducer based on ultrasonic echoes from a bone structure inside the cranium; determining respective delay correction quantities corresponding to each ultrasound transducer based on said determined propagation times; transmitting respective ultrasonic waves toward a subject from each ultrasound transducer while correcting wavefront disorder of the ultrasonic waves arising due to a thickness distribution of the cranium by said determined delay correction quantity; and generating an ultrasound image based on ultrasonic echoes received from the subject.

### BACKGROUND OF THE INVENTION

The present invention relates to an ultrasound imaging method and apparatus which produce ultrasound images, and in particular, to an ultrasound imaging method and apparatus which produce intracranial ultrasound images.

Ultrasound imaging by which ultrasound images are produced by transmitting an ultrasonic beam from an ultrasound probe toward a subject and receiving the ultrasonic echoes reflected by the subject is known. Ultrasound imaging has been applied to observation inside the body. For example, ultrasound images of a subject have been produced by transmitting an ultrasonic beam toward a subject inside the cranium from the temple to the head.

In this type of internal ultrasound imaging, the ultrasonic beam propagates through layered body tissues having different properties. For this reason, there are the problems that these differences in properties of body tissues affect the propagation time of the ultrasonic beam, and the focal point position of the ultrasonic beam with respect to the subject deviates. In particular, in intracranial ultrasound imaging, since the ultrasonic beam propagates through body tissues in which the ultrasound propagation time differs greatly, such as cranial bone and brain tissue, the deviation in focal point position is also large.

Thus, in JP 08-308832 A, for example, a technique was proposed whereby, in internal ultrasound imaging, the speed of the ultrasonic beam is varied so as to form a focal point of the ultrasonic beam at a certain position.

However, in cases where the ultrasonic waves that constitute the ultrasonic beam propagate through body tissues of different thicknesses and so forth, since each ultrasonic wave propagates through the body with a different propagation time, there is the risk of generating disorder of the wavefront even if the speed of the ultrasonic beam is varied and a focal point is formed at a certain position.

### SUMMARY OF THE INVENTION

An object of the present invention is to provide an ultrasound imaging method and ultrasound imaging apparatus for solving these problems of the past, which can suppress wavefront disorder arising due to the fact that the ultrasonic waves that constitute the ultrasonic beam propagate through the body with different propagation times.

An ultrasound imaging method according to a first aspect of the present invention comprises the steps of:

transmitting respective ultrasonic waves from a plurality of ultrasound transducers, arranged in an array, of an ultrasound probe;

determining respective propagation times of the ultrasonic waves inside a cranium corresponding to each ultrasound transducer based on ultrasonic echoes from a bone structure inside the cranium;

determining respective delay correction quantities corresponding to each ultrasound transducer based on said determined propagation times;

transmitting respective ultrasonic waves toward a subject from each ultrasound transducer while correcting wavefront disorder of the ultrasonic waves arising due to a thickness distribution of the cranium by said determined delay correction quantity; and

generating an ultrasound image based on ultrasonic echoes received from the subject.

An ultrasound imaging method according to a second aspect of the present invention comprises the steps of:

transmitting respective ultrasonic waves from a plurality of ultrasound transducers, arranged in an array, of an ultrasound probe;

calculating a correlation of reception signals in each frequency domain received by mutually adjacent ultrasound transducers, for a high-luminance portion that indicates a bone structure inside a cranium at medium depth and beyond of an ultrasound image;

determining a delay correction quantity corresponding to each ultrasound transducer based on the results of said correlation; transmitting respective ultrasonic waves toward a subject from each ultrasound transducer while correcting wavefront disorder of the ultrasonic waves arising due to a thickness distribution of the cranium by said determined delay correction quantity; and

regenerating an ultrasound image based on ultrasonic echoes received from the subject.

An ultrasound imaging apparatus according to a third aspect of the present invention comprises:

an ultrasound probe having a plurality of ultrasound transducers arranged in an array;

an image generator which generates an ultrasonic image based on ultrasonic echoes received by each ultrasonic transducer of said ultrasound probe;

a propagation time calculator which determines the respective propagation times of ultrasonic waves inside a cranium corresponding to each ultrasound transducer based on ultrasonic echoes from a bone structure inside the cranium;

a delay correction quantity calculator which determines respective delay correction quantities corresponding to each ultrasound transducer based on said propagation times determined by said propagation time calculator;

a corrector which corrects wavefront disorder of ultrasonic waves arising due to a thickness distribution of the cranium by said delay correction quantity determined by said delay correction quantity calculator; and

a controller which causes respective ultrasonic waves to be transmitted toward a subject from each ultrasound transducer of said ultrasound probe while wavefront disorder of the ultrasonic waves is corrected by said corrector.

An ultrasound imaging apparatus according to a fourth aspect of the present invention comprises:

an ultrasound probe having a plurality of ultrasound transducers arranged in an array;

an image generator which generates an ultrasonic image based on ultrasonic echoes received by each ultrasonic transducer of said ultrasound probe;

a correlation calculator which calculates a correlation of reception signals in each frequency domain received by mutually adjacent ultrasound transducers, for a high-luminance portion of a bone structure inside a cranium at medium depth and beyond of said ultrasound image;

a delay correction quantity calculator which determines delay correction quantities corresponding to each ultrasound transducer based on the results of correlation by said correlation calculator;

a corrector which corrects wavefront disorder of ultrasonic waves arising due to a thickness distribution of the cranium by said delay correction quantity determined by said delay correction quantity calculator; and

a controller which causes respective ultrasonic waves to be transmitted toward a subject from each ultrasound transducer of said ultrasound probe while wavefront disorder of the ultrasonic waves is corrected by said corrector.

### BRIEF DESCRIPTION OF THE DRAWINGS

FIG. 1 is a block diagram illustrating a configuration of an ultrasound imaging apparatus according to embodiment 1 of the present invention.

FIG. 2 is a drawing illustrating an arrangement position of an ultrasound probe used in embodiment 1.

FIG. 3 is a drawing illustrating a distribution of thickness of a cranium for ultrasound transducers.

FIG. 4 is a drawing illustrating the position at which an ultrasonic wave propagates through the cranium.

FIG. 5 is a drawing illustrating the difference in propagation times at which ultrasonic waves propagate through the cranium.

FIG. 6 is a drawing illustrating the state where a delay correction quantity is determined from the propagation times.

FIG. 7 is a drawing illustrating the state where an ultrasonic wave transmitted using a corrected delay instruction quantity propagates through the cranium.

FIG. 8 is a drawing illustrating the state where an ultrasonic wave transmitted using an uncorrected delay instruction quantity propagates through the cranium.

FIG. 9 is a drawing illustrating wavefronts of the ultrasonic waves transmitted from the ultrasound transducers.

FIG. 10 is a drawing illustrating waveforms of the echoes received by the ultrasound transducers in embodiment 2.

FIG. 11 is a drawing illustrating the state where a propagation time calculator calculates the propagation time of an ultrasonic wave inside the cranium in embodiment 2.

FIG. 12 is a perspective view illustrating the arrangement direction of ultrasonic generators used in embodiment 3.

FIG. 13 is a top view illustrating the arrangement direction of the ultrasonic generators used in embodiment 3.

FIG. 14 is a drawing illustrating the state where an aperture width of each ultrasonic generator used in embodiment 3 is adjusted.

FIG. 15 is a block diagram illustrating a configuration of an ultrasound imaging apparatus according to embodiment 4.

FIG. 16 is a flow chart representing the operation of the ultrasound imaging apparatus according to embodiment 4.

FIG. 17 shows an ultrasound image before wavefront correction in embodiment 4.

FIG. 18 shows an ultrasound image after wavefront correction has been performed in embodiment 4.

### DETAILED DESCRIPTION OF THE INVENTION

The present invention is described in detail hereinafter based on the preferred embodiments shown in the accompanying drawings.

### Embodiment 1

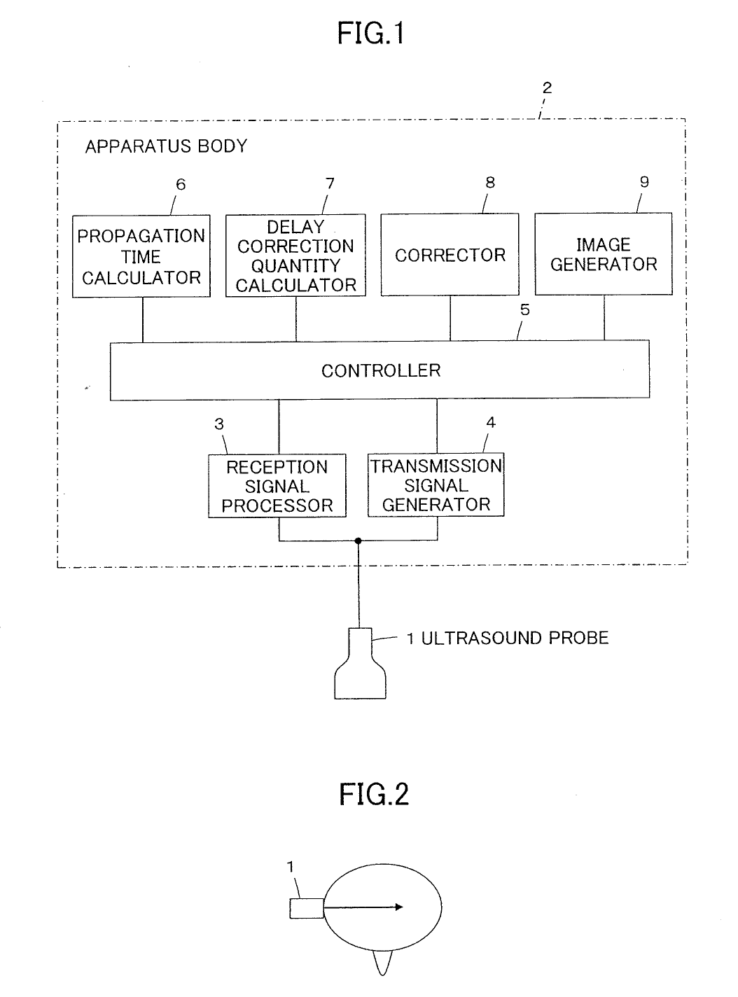

FIG. 1 illustrates a configuration of the ultrasound imaging apparatus according to embodiment 1 of the present invention. The ultrasound imaging apparatus comprises an ultrasound probe 1 and an apparatus body 2.

The ultrasound probe 1 comprises an ultrasound probe element which contains a plurality of ultrasound transducers arranged in an array. The apparatus body 2 comprises a reception signal processor 3 and a transmission signal generator 4, which are connected to the ultrasound probe 1. Reception signals corresponding to the ultrasonic echoes received by the ultrasound probe 1 is input from the ultrasound probe 1 to the reception signal processor 3. The transmission signal generator 4 generates transmission signals and outputs them to the ultrasound probe 1.

A controller 5 is connected to the reception signal processor 3 and the transmission signal generator 4. The controller 5 controls the input and output of signals to and from the parts in the apparatus body 2.

Additionally, a propagation time calculator 6, a delay correction quantity calculator 7, a corrector 8 and an image generator 9 are each connected to the controller 5. The propagation time calculator 6 determines the respective propagation times for which the ultrasonic waves received by the ultrasound transducers of the ultrasound probe element of the ultrasound probe 1 propagate inside the cranium, based on the ultrasonic echoes from the bone structure inside the cranium. The delay correction quantity calculator 7 determines the respective delay correction quantities for correcting the delay instruction quantities which indicate the transmission timing of ultrasonic waves transmitted from the ultrasound transducers, based on the propagation time determined by the propagation time calculator 6. The corrector 8 corrects wavefront disorder of the ultrasonic beam occurring due to the thickness distribution of the cranium by correcting the delay instruction quantity with the delay correction quantity determined by the delay correction quantity calculator 7. The image generator 9 generates an ultrasound image based on the reception signals corresponding to the ultrasonic echoes received by the reception signal processor 4.

Next, the operation of the ultrasound imaging apparatus illustrated in FIG. 1 will be described.

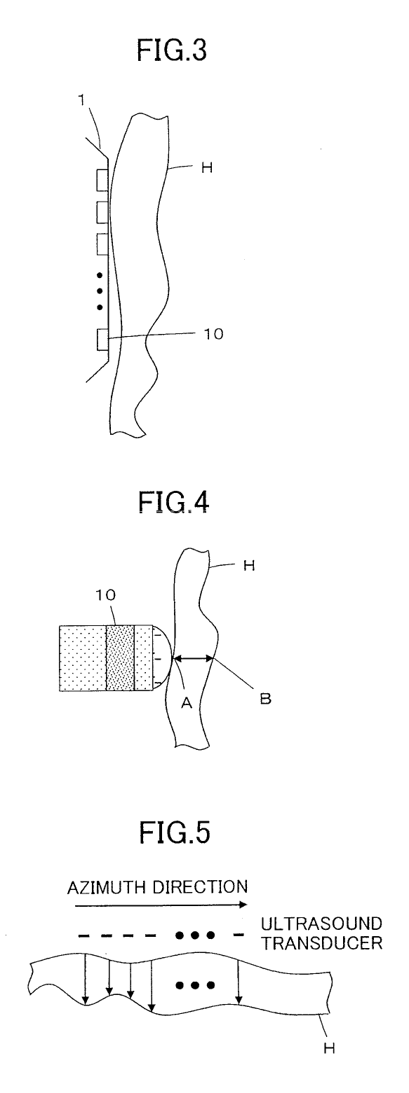

First, as shown in FIG. 2, the ultrasound probe 1 is arranged at a prescribed position on the head. In the ultrasound probe element of the arranged ultrasound probe 1, as shown in FIG. 3, ultrasound transducers 10 for transmitting ultrasonic waves are arranged in a straight line, whereas the outer surface and inner surface of the cranium H has a curved form. For this reason, the corresponding thickness of the cranium H differs depending on the position of the ultrasound transducer 10 in the ultrasound probe 1. Here, in order to identify the thickness distribution of the cranium H, one frame of reception signals is acquired by transmitting an ultrasonic wave from each ultrasound transducer toward the cranium H.

That is, as shown in FIG. 4, the ultrasonic wave transmitted from each ultrasound transducer 10 of the ultrasound probe 1 reaches point A on the outer surface of the cranium H, and an ultrasonic echo Ea, which is reflected at point A and returned, and an ultrasonic echo Eb, which passes through point A and propagates inside the cranium H and is then reflected at point B on the inner surface of the cranium H, are received by each ultrasound transducer 10. Here, as shown in FIG. 5, a difference arising due to a difference in thickness of the cranium H corresponding to each ultrasound transducer 10 is produced during the time from when the ultrasonic echo Ea is received until the ultrasonic echo Eb is received.

The reception signals of ultrasonic echoes Ea and Eb received by each transducer 10 of the ultrasound probe 1 are input into the reception signal processor 4. These reception signals are transmitted to the propagation time calculator 6 via the controller 5.

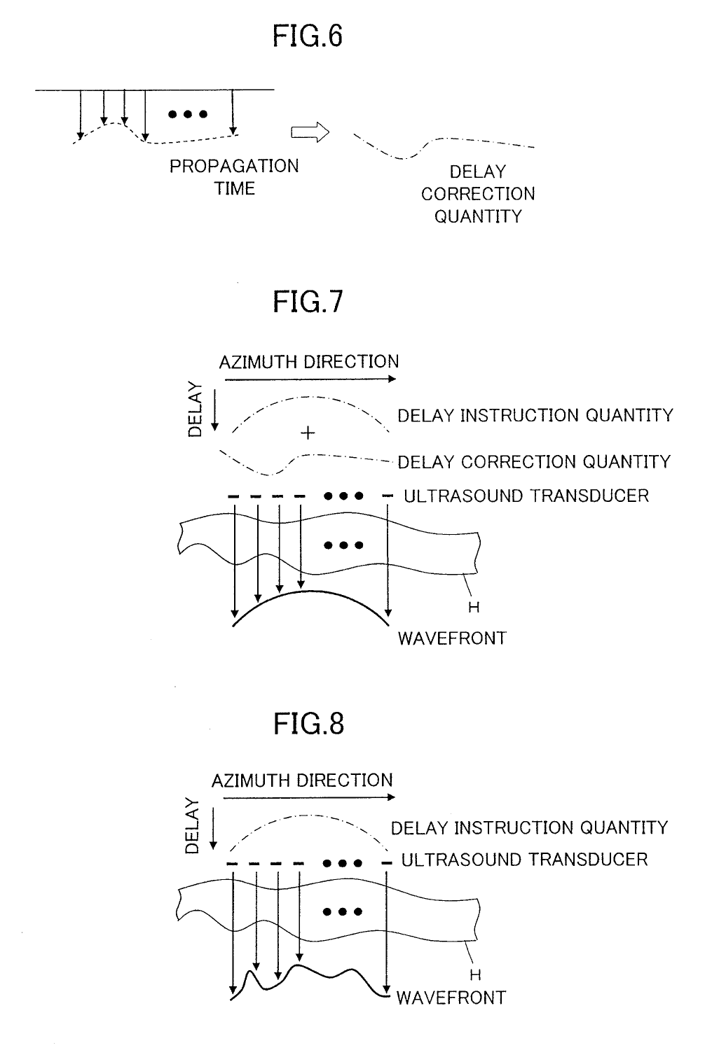

Based on the reception signals input from the reception signal processor 3 via the controller 5, the propagation time calculator 6 calculates the respective propagation time required for the ultrasonic wave transmitted from each ultrasound transducer 10 arranged in a straight line in the azimuth direction in the ultrasound probe 1 to propagate through the cranium H. That is, based on the time of reception of the ultrasonic echoes Ea and Eb in each ultrasound transducer 10, the propagation time calculator 6 calculates the respective time from when the ultrasonic wave transmitted from each ultrasound transducer 10 reaches the corresponding point A on the cranium H until it reaches point B on the cranium H, as shown in FIG. 6. The calculated propagation time through the cranium H of the ultrasonic wave transmitted from each ultrasound transducer 10 is output from the propagation time calculator 6 to the controller 5, and the controller 5 outputs it to the delay correction quantity calculator 7.

Based on the propagation time received from the propagation time calculator 6 via the controller 5, the delay correction quantity calculator 7 calculates the delay correction quantity that corrects the delay instruction quantity of each ultrasound transducer 10 such that the ultrasonic waves transmitted from the ultrasound transducers 10 of the ultrasound probe 1 form a uniform wavefront after exiting the cranium H, regardless of the thickness distribution of the cranium H. The calculated delay correction quantity of each ultrasound transducer 10 is output from the delay correction quantity calculator 7 to the controller 5, and the controller 5 outputs it to the corrector 8.

As shown in FIG. 7, the corrector 8 performs wavefront correction of the ultrasonic beam by applying the delay correction quantity of each ultrasound transducer 10 received from the delay correction quantity calculator 7 via the controller 5 to the delay instruction quantity of the ultrasonic wave transmitted from each ultrasound transducer 10. The corrected delay instruction quantity is output from the corrector 8 to the controller 5, and the controller 5 outputs it to the transmission signal generator 4.

In this way, the transmission signal generator 4 outputs transmission signals corresponding to the delay instruction quantity corrected by the corrector 8 to each ultrasound transducer 10 of the ultrasound probe 1, and an ultrasonic wave is transmitted from each ultrasound transducer 10. The ultrasonic wave transmitted from each ultrasound transducer 10 propagates through the cranium H, and the difference in propagation times arising due to a thickness distribution of the cranium occurring here is cancelled out by the delay correction quantity, and after the ultrasonic waves propagate through the cranium H, an ultrasonic beam wavefront that is unaffected by the thickness distribution of the cranium H is obtained.

As shown in FIG. 8, if an ultrasonic wave from each ultrasound transducer 10 are transmitted using only the delay instruction quantity without taking the thickness of the cranium H into consideration, ultrasonic beam wavefront disorder arising due to the thickness distribution of the cranium H occurs on the wavefront of the ultrasonic beam after propagating through the cranium H. In contrast, as shown in FIG. 7, ultrasonic beam wavefront disorder after propagating through the cranium H can be corrected due to the fact that the corrector 8 corrects the delay instruction quantity by the delay correction quantity.

The ultrasonic beam that propagated through the cranium H reaches the subject inside the cranium H, and the ultrasonic echoes reflected by the subject are received by each ultrasound transducer 10 of the ultrasound probe 1. The reception signals of ultrasonic echoes from the subject received by the ultrasound probe 1 are input into the reception signal processor 4. When the reception signal processor 4 outputs reception signals corresponding to the input ultrasonic echoes to the controller 5, the controller 5 outputs those reception signals to the image generator 9, and the image generator 9 generates an ultrasound image based on the input signals.

According to the ultrasound imaging apparatus of this embodiment, ultrasonic beam wavefront disorder arising due to the thickness distribution of the cranium H can be corrected.

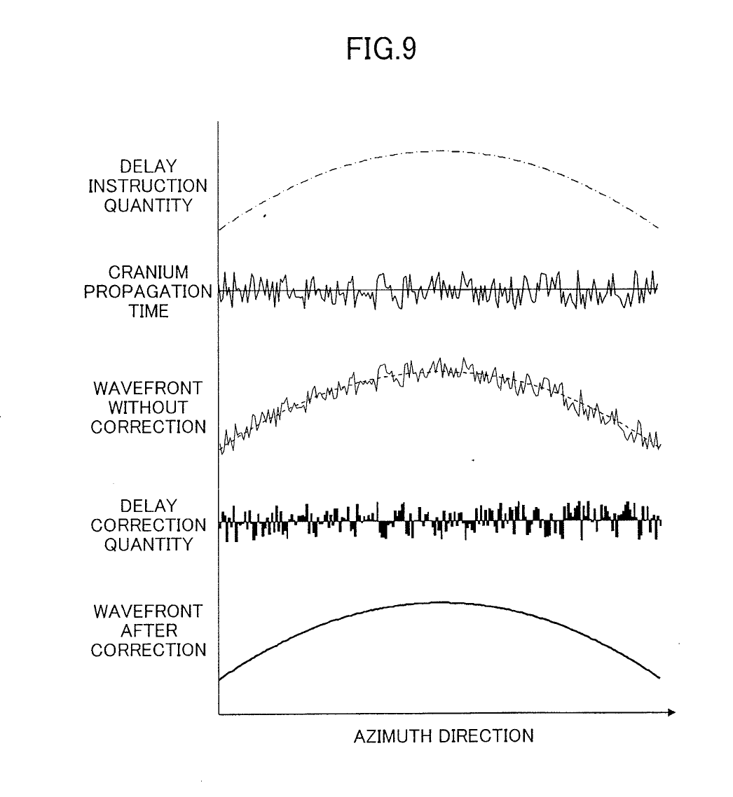

Here, an example in which ultrasonic beam wavefront disorder arising due to the thickness distribution of the cranium H is corrected will be described. As shown in FIG. 9, for each ultrasonic wave transmitted using a certain delay instruction quantity from each ultrasound transducer 10 arranged in the azimuth direction of the ultrasound probe 1, the propagation time required for each ultrasonic wave to propagate through the cranium H is respectively obtained by the propagation time calculator 6. If ultrasonic waves from each ultrasound transducer 10 are transmitted using only the delay instruction quantity without taking the thickness of the cranium H into consideration, ultrasonic beam wavefront disorder arising due to the thickness distribution of the cranium H occurs on the wavefront of the ultrasonic beam after each ultrasonic wave propagates through the cranium H.

On the other hand, the delay correction quantity calculator 7 determines the delay correction quantity based on the propagation time of each ultrasonic wave through the cranium H obtained by the propagation time calculator 6, and the corrector 8 corrects the delay instruction quantity based on the delay correction quantity, and an ultrasonic wave is transmitted from each ultrasound transducer 10 using the delay instruction quantity corrected by the corrector 8. As a result, the ultrasonic waves transmitted from the ultrasound transducers 10 form an ultrasonic beam wavefront that is unaffected by the thickness distribution of the cranium H after propagating through the cranium H.

### Embodiment 2

The propagation time of an ultrasonic wave inside the cranium H can also be calculated by adding the multiple ultrasonic echoes from the bone structure inside the cranium H in each frequency domain.

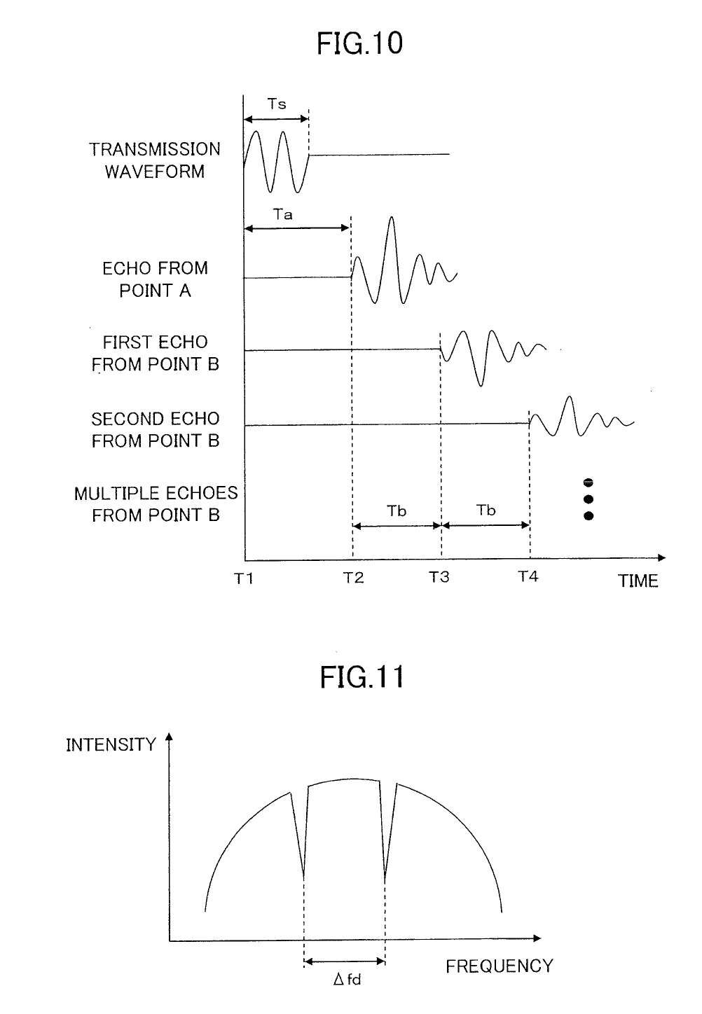

As shown in FIG. 10, when an ultrasonic wave is transmitted at time T1 from the ultrasound transducer 10 of the ultrasound probe 1, the transmitted ultrasonic wave reaches point A on the outer surface of the cranium H, and the ultrasonic echo reflected at point A on the cranium H and returned is received at time T2 by the ultrasound transducer 10. Here, in the ultrasound transducer 10, the ultrasonic wave is generated by an ultrasonic generator made from PZT (lead zirconate titanate) or the like, and it is transmitted by propagating through an ultrasound converging portion made from an acoustic lens or the like. For this reason, the ultrasonic echo from point A on the cranium H is received with a delay of propagation time Ta inside the ultrasound transducer 10 from time T1 of transmission.

On the other hand, the ultrasonic wave transmitted from the ultrasound transducer 10 which passed through point A on the cranium H propagates through the cranium H, and a first echo reflected at point B on the inner surface of the cranium H is received by the ultrasound transducer 10 at time T3. Additionally, after the first echo is reflected at point A on the cranium H, a second echo reflected again at point B on the cranium H is received by the ultrasound transducer 10 at time T4. Similarly, multiple echoes from point B on the cranium H are received in sequence by the ultrasound transducer 10. Here, the duration from time T2 to time T3, the duration from time T3 to time T4, etc. is the propagation time Tb required for the first echo, second echo, etc., respectively, to propagate through the cranium H.

For example, when an ultrasonic wave of frequency 2 MHz and wave number n=2 is transmitted with a transmission time of Ts=1.0 μs toward a cranium H of thickness 2 mm, the ultrasonic wave propagates inside the ultrasound transducer 10 with a propagation time of Ta=2-3 μs, and it propagates through the cranium H with a propagation time of Tb=1.2 μs, and is received by the ultrasound transducer 10.

In this way, if the first echo, second echo, etc. from point B on the cranium H received by the ultrasound transducer 10 and the transmitted ultrasonic wave are processed by fast Fourier transform (FFT) and added in every frequency domains, for example, as shown in FIG. 11, a notch occurs with a frequency gap Δfd corresponding to a gap in the multiple echoes from point B on the cranium H, that is, propagation time Tb required for the first echo, second echo, etc. to respectively propagate through the cranium H. This is because the ultrasonic echo from point A on the cranium H and the ultrasonic echo from point B on the cranium H have the relationship of a comb filter, and a notch or peak occurs with a frequency gap given by the reciprocal number of the time difference of reception (propagation time Tb) of the ultrasonic echoes from point A and point B on the cranium by the ultrasound transducer 10.

Thus, the propagation time calculator 6 can determine the propagation time of the ultrasonic wave inside the cranium H from the notch or peak frequency gap Δfd based on Tb=1/Δfd. Similarly, it determines the propagation times inside the cranium H for the ultrasonic waves transmitted from all of the ultrasound transducers 10. Using the propagation time of each ultrasonic wave determined in this way, the delay correction quantity calculator 7 can determine the delay correction quantity, and the corrector 8 can correct the delay instruction quantity based on the delay correction quantity.

According to embodiment 2, the load can be reduced when the propagation time calculator 6 determines the propagation times of the ultrasonic waves inside the cranium H.

### Embodiment 3

The ultrasound transducer 10 of the ultrasound probe 1 used in embodiments 1 and 2 comprises ultrasonic generators made from PZT or the like, but by adjusting the aperture width of the ultrasonic generators, the near field length of the ultrasonic wave transmitted from each ultrasound transducer 10 can be set close to point B on the inner surface of the cranium H.

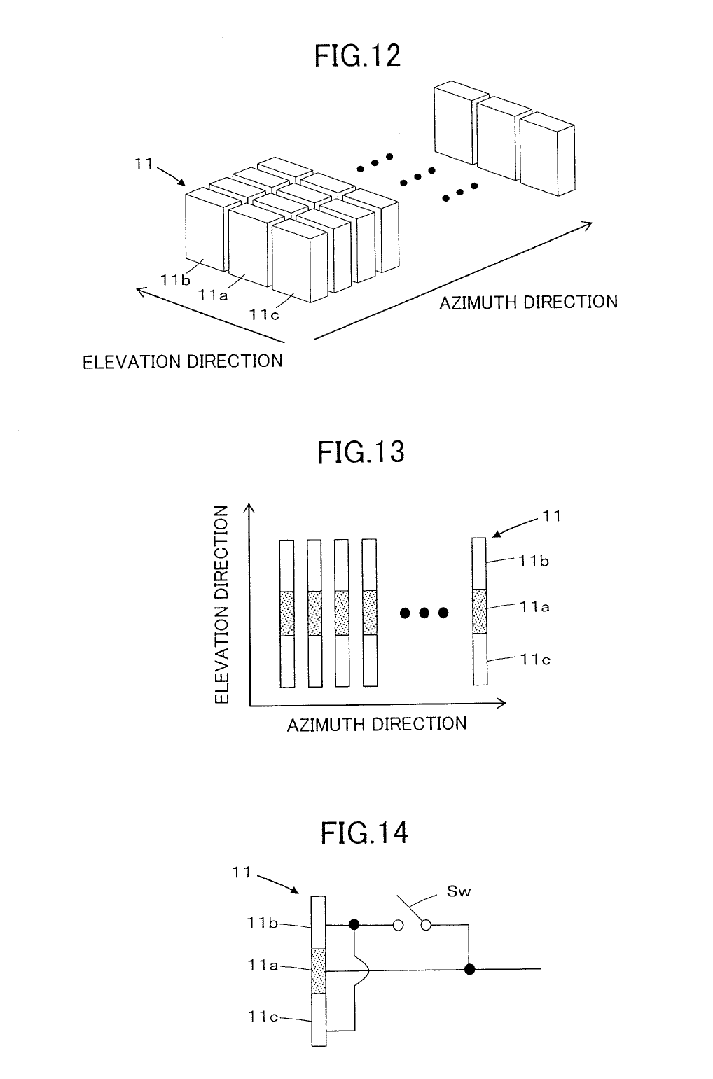

For example, as shown in FIG. 12, by arranging ultrasonic generators 11 in the azimuth direction and dividing them into a plurality of elements in the elevation direction, the aperture width of each ultrasonic generator 11 can be adjusted. As shown in FIG. 13, the ultrasonic generators 11 may be divided into three elements, first element 11a, second element 11b and third element 11c, in the elevation direction, where the first element 11a is arranged in the center and the second element 11b and third element 11c are arranged on the two sides of the first element 11a. As shown in FIG. 14, the first element 11a is connected with the second element 11b and third element 11c such that the first element 11a can be electrically connected to or disconnected from the second element 11b and third element 11c by switching a switch Sw.

When the propagation time calculator 6 determines the propagation time of an ultrasonic wave inside the cranium H, it uses the narrow aperture width of only the first element 11a by opening the switch Sw. As a result, the near field length of the ultrasonic wave transmitted from each ultrasound transducer 10 is close to point B on the inner surface of the cranium H, and the propagation time is determined based on the ultrasonic echoes from the bone structure in the cranium H. Also, when a subject inside the cranium H is imaged, the propagation time calculator 6 uses a wide aperture width using the first element 11a, second element 11b and third element 11c by closing the switch Sw. As a result, the near field length of the ultrasonic wave transmitted from each ultrasound transducer 10 is close to the subject in the cranium H, and an ultrasound image is produced based on the ultrasonic echoes from the subject inside the cranium H.

According to embodiment 3, a subject inside the cranium H can be imaged with high precision by means of the propagation time calculator 6 adjusting the aperture width of the ultrasonic generator 11 in accordance with the distance between the object toward which ultrasonic waves are transmitted and the ultrasonic generator 11.

### Embodiment 4

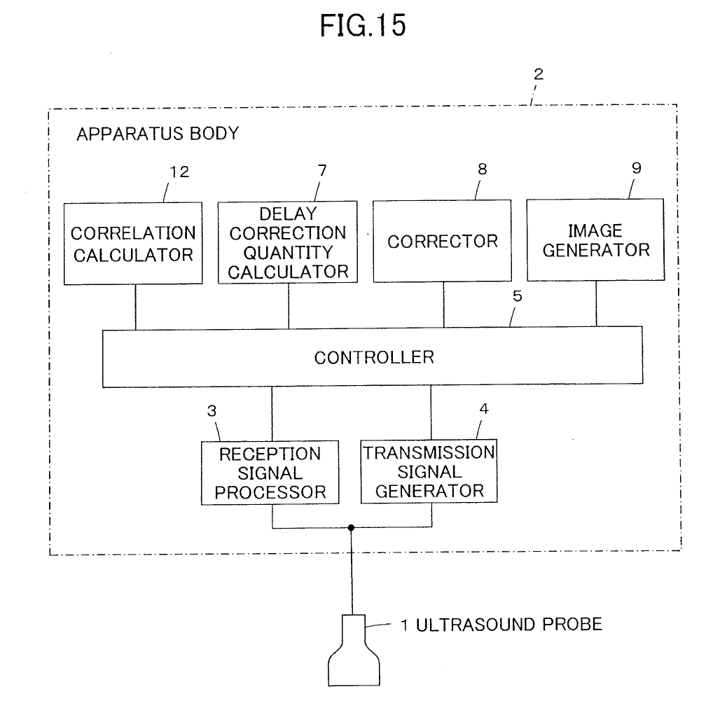

FIG. 15 illustrates a configuration of an ultrasound imaging apparatus according to embodiment 4. This ultrasound imaging apparatus uses an apparatus body 13 in which a correlation calculator 12 is connected to the controller 5, instead of the apparatus body 2 in which the propagation time calculator 6 is connected to the controller 5 as in the apparatus of embodiment 1 shown in FIG. 1. The other members are the same as in the apparatus of embodiment 1 shown in FIG. 1.

The correlation calculator 12 calculates the correlation of the reception signals in each frequency domain received by mutually adjacent ultrasound transducers 10 of the ultrasound probe element of the ultrasound probe 1, for the high-luminance portion that indicates the cranial bone structure at medium depth and beyond of the ultrasound image. In embodiment 4, the delay correction quantity calculator 7 calculates the delay correction quantity corresponding to each transducer 10 based on the correlation results calculated by the correlation calculator 12.

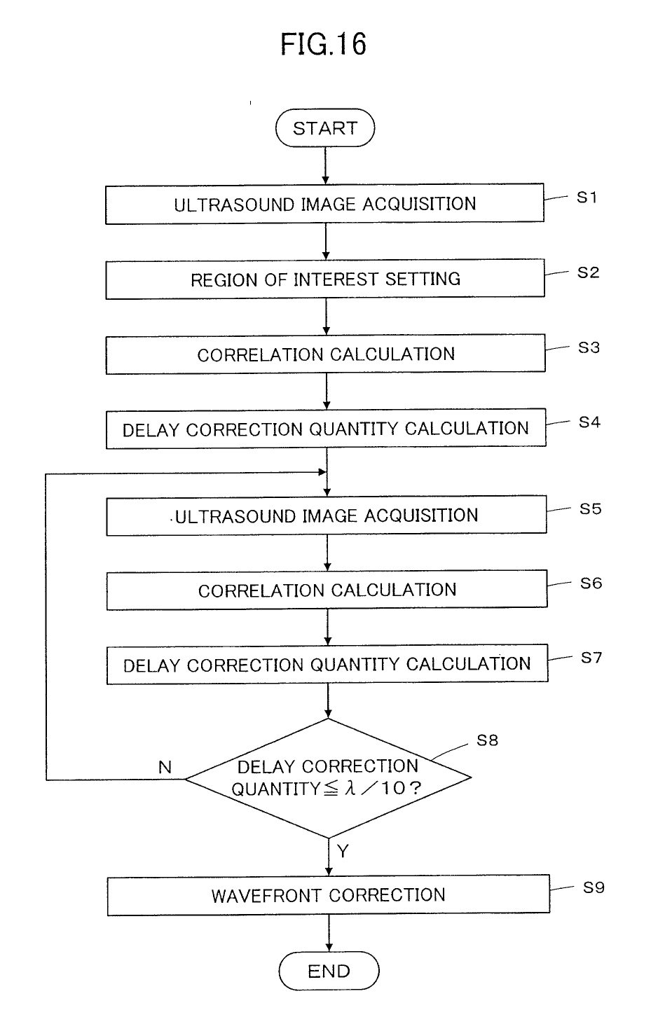

The ultrasound imaging method in embodiment 4 will be described referring to the flow chart of FIG. 16.

First, in step S1, one frame of ultrasound image of a subject is acquired. That is, according to an instruction from the controller 5, an ultrasonic beam is transmitted toward the subject from the ultrasound probe 1 based on the transmission signal generated by the transmission signal generator 4, and the ultrasonic echoes received by the ultrasound probe 1 are processed by the reception signal processor 3. After that, the reception signal is sent to the image generator 9 via the controller 5, and one frame of ultrasound image (B mode image) is generated by the image generator 9.

In step S2, the high-luminance portion of medium depth and beyond of this ultrasound image is identified by the correlation calculator 12, and it is set as the region of interest R. For example, the high-luminance portion due to the ultrasonic echoes from the sphenoid bone or cranium on the side opposite the side where the ultrasound probe 1 is arranged may be set as the region of interest R.

Then, in step S3, the correlation calculator 12 calculates the correlation for the reception signal of the ultrasound probe 1 corresponding to the region of interest R. That is, phase matching is performed, in which a prescribed delay quantity is provided between the reception signals received by all ultrasound transducers 10 of the ultrasound probe 1 in the scan line direction in which the high-luminance ultrasonic echoes are obtained and the reception signal received by the respective adjacent ultrasound transducer 10, and the S/N ratio of the addition signal obtained by adding the phase-matched reception signals to each other is calculated.

Additionally, the correlation calculator 12 performs respective phase matching while variously changing the prescribed delay quantity, and calculates the S/N ratio of the addition signal.

In step S4, the delay correction quantity calculator 7 compares the S/N ratios of the respective addition signals calculated by the correlation calculator 12, and when the S/N ratio reaches its maximum, it is judged that the correlation between reception signals is best and the azimuth resolution is best, and the delay quantity given when the addition signal having this maximum S/N ratio is obtained is calculated as the delay correction quantity of that ultrasound transducer 10. Similarly, the delay correction quantities of all ultrasound transducers 10 of the ultrasound probe 1 are calculated.

Next, in step S5, according to an instruction from the controller 5, the respective ultrasonic wave is transmitted from each ultrasound transducer 10 of the ultrasound probe 1 based on the delay correction quantity calculated in step S4 (that is, taking the delay correction quantity into consideration), and the ultrasonic echoes are received, and one frame of ultrasound image is again acquired.

Additionally, in step S6, similar to step S3 described above, the correlation calculator 12 calculates the correlation for the reception signal of the ultrasound probe 1 corresponding to the region of interest R. Then, in step S7, similar to step S4 described above, the delay correction quantity calculator 7 calculates the delay correction quantity of each ultrasound transducer 10 of the ultrasound probe 1.

In step S8, the controller 5 judges whether or not the delay correction quantity computed in step S7 is within λ/10 with respect to the wavelength λ transmitted and received by the ultrasound probe 1. As a result of this judgment, if the delay correction quantity is not within λ/10, it is judged that the delay correction quantity must be modified, and it returns to step S5, where one frame of ultrasound image is acquired based on the delay correction quantity calculated in step S7, and then correlation is calculated in step S6, and the delay correction quantity is calculated in step S7. Steps S5 through S8 are repeated in this way until the delay correction quantity is within λ/10.

When it is judged in step S8 that the delay correction quantity is within λ/10, it is judged that an appropriate delay correction quantity has been obtained, and the process proceeds to step S9, where the ultrasonic beam wavefront disorder arising from the thickness distribution of the cranium is corrected by the corrector 8 using this delay correction quantity. The ultrasonic beam of which the wavefront disorder has been corrected is transmitted, and the obtained ultrasound image is generated by the image generator 9 and displayed on a display monitor or the like.

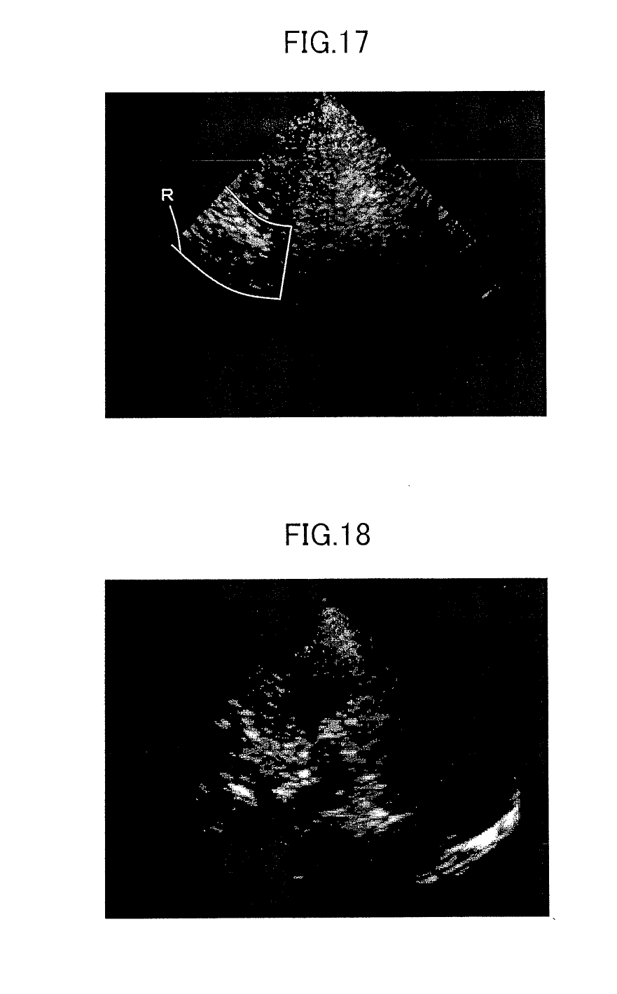

An example in which wavefront correction was actually performed according to embodiment 4 is shown in FIG. 17 and FIG. 18. FIG. 17 shows the ultrasound image before correction in which the region of interest R was set. When wavefront correction was performed on this ultrasound image according to embodiment 4, the ultrasound image shown in FIG. 18 was obtained. It can be confirmed that the image was clearer than before correction.

Note that an example of the method of ultrasound image acquisition employed in step S1 can be a method wherein ultrasonic wave transmission and reception are performed 10 times immediately after the frame begins, and the reception signals are collected and stored in memory, and after that, transmission and reception of ultrasonic waves is paused, and an ultrasound image is constructed using all of the reception signals stored in memory during that time. If such a method is used, because the collection time of ultrasonic echoes is shorter than the frame rate, temporal simultaneity within one frame image is good, and a clearer image can be obtained. In addition, because ultrasonic echo data can be collected with a small number of transmissions, there is the advantage that a drop in the wide frame rate does not occur even in color Doppler, triplex mode and the like.

In embodiment 4, similar to embodiment 1, wavefront disorder of ultrasonic waves due to thickness distribution of the cranium H can be corrected.

What is claimed is:

1. An ultrasound imaging method comprising the steps of:
transmitting respective ultrasonic waves from a plurality of ultrasound transducers, arranged in an array, of an ultrasound probe;
determining respective propagation times of the ultrasonic waves inside a cranium corresponding to each ultrasound transducer based on ultrasonic echoes from a bone structure inside the cranium;
determining respective delay correction quantities corresponding to each ultrasound transducer based on said determined propagation times;
transmitting respective ultrasonic waves toward a subject from each ultrasound transducer while correcting wavefront disorder of the ultrasonic waves arising due to a thickness distribution of the cranium by said determined delay correction quantity; and
generating an ultrasound image based on ultrasonic echoes received from the subject.

2. The ultrasound imaging method according to claim 1, wherein the propagation times of the ultrasonic waves inside the cranium are measured by transmitting an ultrasonic wave from each ultrasound transducer while adjusting an aperture width of each ultrasound transducer such that a near field length is close to the inner surface of the cranium.

3. The ultrasound imaging method according to claim 2, wherein: each ultrasound transducer is divided into a plurality of portions in an elevation direction thereof; and said aperture width is adjusted by electrically connecting and disconnecting the divided plurality of portions to and from each other.

4. The ultrasound imaging method according to claim 1, wherein said propagation times of ultrasonic waves inside the cranium are calculated by analyzing multiple ultrasonic echoes from the bone structure inside the cranium in each frequency domain.

5. An ultrasound imaging method comprising the steps of:
transmitting respective ultrasonic waves from a plurality of ultrasound transducers, arranged in an array, of an ultrasound probe;
calculating a correlation of reception signals in each frequency domain received by mutually adjacent ultrasound transducers, for a high-luminance portion that indicates a bone structure inside a cranium at medium depth and beyond of an ultrasound image;
determining a delay correction quantity corresponding to each ultrasound transducer based on the results of said correlation;
transmitting respective ultrasonic waves toward a subject from each ultrasound transducer while correcting wavefront disorder of the ultrasonic waves arising due to a thickness distribution of the cranium by said determined delay correction quantity; and
regenerating an ultrasound image based on ultrasonic echoes received from the subject.

6. The ultrasound imaging method according to claim 5, wherein:
a delay quantity is provided between the reception signals in each frequency domain received by mutually adjacent transducers, and respective phase matching is performed by changing said delay quantity; and
the delay quantity of when azimuth resolution is best is used as said delay correction quantity.

7. An ultrasound imaging apparatus comprising:
an ultrasound probe having a plurality of ultrasound transducers arranged in an array;
an image generator which generates an ultrasonic image based on ultrasonic echoes received by each ultrasonic transducer of said ultrasound probe;
a propagation time calculator which determines the respective propagation times of ultrasonic waves inside a cranium corresponding to each ultrasound transducer based on ultrasonic echoes from a bone structure inside the cranium;
a delay correction quantity calculator which determines respective delay correction quantities corresponding to each ultrasound transducer based on said propagation times determined by said propagation time calculator;
a corrector which corrects wavefront disorder of ultrasonic waves arising due to a thickness distribution of the cranium by said delay correction quantity determined by said delay correction quantity calculator; and
a controller which causes respective ultrasonic waves to be transmitted toward a subject from each ultrasound transducer of said ultrasound probe while wavefront disorder of the ultrasonic waves is corrected by said corrector.

8. The ultrasound imaging apparatus according to claim 7, wherein said propagation time calculator measures the propagation times of the ultrasonic waves inside the cranium by transmitting an ultrasonic wave from each ultrasound transducer while adjusting an aperture width of each ultrasound transducer such that a near field length is close to the inner surface of the cranium.

9. The ultrasound imaging apparatus according to claim 8, wherein each ultrasound transducer is divided into a plurality of portions in an elevation direction thereof, and said propagation time calculator adjusts said aperture width by electrically connecting and disconnecting the divided plurality of portions to and from each other.

10. The ultrasound imaging apparatus according to claim 7, wherein said propagation time calculator calculates said propagation times of ultrasonic waves inside the cranium by analyzing multiple ultrasonic echoes from the bone structure inside the cranium in each frequency domain.

11. An ultrasound imaging apparatus comprising:
an ultrasound probe having a plurality of ultrasound transducers arranged in an array;
an image generator which generates an ultrasonic image based on ultrasonic echoes received by each ultrasonic transducer of said ultrasound probe;
a correlation calculator which calculates a correlation of reception signals in each frequency domain received by mutually adjacent ultrasound transducers, for a high-luminance portion of a bone structure inside a cranium at medium depth and beyond of said ultrasound image;
a delay correction quantity calculator which determines delay correction quantities corresponding to each ultrasound transducer based on the results of correlation by said correlation calculator;
a corrector which corrects wavefront disorder of ultrasonic waves arising due to a thickness distribution of the cranium by said delay correction quantity determined by said delay correction quantity calculator; and
a controller which causes respective ultrasonic waves to be transmitted toward a subject from each ultrasound transducer of said ultrasound probe while wavefront disorder of the ultrasonic waves is corrected by said corrector.

12. The ultrasound imaging apparatus according to claim 11, wherein:
said correlation calculator provides a delay quantity between the reception signals in each frequency domain received by mutually adjacent transducers, and performs respective phase matching by changing said delay quantity; and
said delay correction quantity calculator uses the delay quantity of when azimuth resolution is best as said delay correction quantity.
